(19) United States
(12) Patent Application Publication
(10) Pub. No.: US 2017/0191941 A1
(43) Pub. Date: Jul. 6, 2017

(54) TESTING UNIT, SAMPLE ANALYZER, AND TESTING METHOD

(71) Applicant: Sysmex Corporation, Kobe-shi (JP)

(72) Inventors: Atsushi HOSOTANI, Osaka-shi (JP); Daigo TAKAHASHI, Osaka-shi (JP); Motohiro ODA, Osaka-shi (JP)

(73) Assignee: Sysmex Corporation, Kobe-shi (JP)

(21) Appl. No.: 15/461,745

(22) Filed: Mar. 17, 2017

Related U.S. Application Data
(63) Continuation of application No. PCT/JP2015/076712, filed on Sep. 18, 2015.

(30) Foreign Application Priority Data
Sep. 20, 2014 (JP) 2014-192042

(51) Int. Cl. G01N 21/77; G01N 33/493

(52) U.S. Cl. CPC G01N 21/77; G01N 33/493; G01N 2201/12; G01N 2021/7769

(57) ABSTRACT

A testing unit to test a liquid sample applied on a measurement surface in an indicator section on a test piece by using a brightness distribution. The testing unit comprises an irradiation part that irradiates the measurement surface with light; an imaging part that obtains images of the measurement surface; a generating part that generates a determination index based on the imaging data; and a determination part that executes determination on a measurement item, using the determination index. The determination index is based on a brightness of the measurement surface when the measurement surface does not include a specular reflection region. The determination index is based on a brightness of a remnant region of the measurement surface excluding the specular reflection region when it is determined that the measurement surface includes a specular reflection region.

### CROSS REFERENCE TO RELATED APPLICATIONS

This application is a continuation application of International Application No. PCT/JP2015/076712, filed on Sep. 18, 2015, entitled “TESTING UNIT, SAMPLE ANALYZER, AND TESTING METHOD”, which claims priority based on the Article 8 of Patent Cooperation Treaty from prior Japanese Patent Applications No. 2014-192042, filed on Sep. 20, 2014, the entire contents of which are incorporated herein by reference.

### BACKGROUND

The disclosure relates to a testing unit, a sample analyzer including the testing unit, and a testing method.

There has heretofore been known a method of determining a color condition of a reagent section (indicator section) provided in a test specimen (test piece) by image processing in the case of analyzing concentrations of components contained in a sample such as urine (for example, Japanese Patent No. 3559975 (Patent Literature 1)).

### SUMMARY

One or more embodiments of a testing unit to test a liquid sample applied on a measurement surface in an indicator section on a test piece by using a brightness distribution may comprise: (a) an irradiation part that irradiates a reference surface and the measurement surface on the test piece with light; (b) an imaging part that obtains images of the reference surface and the measurement surface on the test piece; (c) a generating part that generates a determination index based on the imaging data obtained by the imaging part; and (d) a determination part that executes determination, using the determination index, on a measurement item associated with the indicator section, wherein the generating part includes: (c-1) a first calculator that calculates a brightness, as a reference brightness, of the reference surface on the test piece, and calculates a brightness, as a first sample brightness, of the measurement surface, (c-2) a decision part that determines whether or not the imaging data concerning the measurement surface includes a specular reflection region on the basis of a relationship between a first rate calculated as a ratio of the first sample brightness to the reference brightness and a standard deviation or dispersion of a brightness distribution on the measurement surface, and (c-3) a second calculator that calculates a brightness, as a second sample brightness, of a remnant region of the measurement surface excluding the specular reflection region, when the decision part determines that the specular reflection region is present, and the determination part executes the determination on the measurement item associated with the indicator section, (i) using the determination index based on the second sample brightness, when it is determined that the specular reflection region is present, and (ii) using the determination index based on the first sample brightness, when it is determined that the specular reflection region is not present.

One or more embodiments of a testing method for testing a liquid sample applied on a measurement surface in an indicator section on a test piece by using a brightness distribution may comprise: irradiating a reference surface and the measurement surface on the test piece with light; obtaining images of the reference surface and the measurement surface on the test piece; generating a determination index based on the imaging data obtained; and executing determination on a measurement item associated with the indicator section by using the determination index, wherein the generating the determination index includes: (a) calculating a brightness, as a reference brightness, of the reference surface on the test piece, and calculating a brightness, as a first sample brightness, of the measurement surface, (b) determining whether or not the imaging data concerning the measurement surface includes a specular reflection region on the basis of a relationship between a first rate calculated as a ratio of the first sample brightness to the reference brightness and a standard deviation or dispersion of a brightness distribution on the measurement surface, and (c) calculating a brightness, as a second sample brightness, of a remnant region of the measurement surface excluding the specular reflection region, when it is determined that the specular reflection region is present, and the executing the determination on the measurement item includes: (i) executing the determination on the measurement item by using the determination index based on the second sample brightness, when it is determined that the specular reflection region is present, and (ii) executing the determination on the measurement item by using the determination index based on the first sample brightness, when it is determined that the specular reflection region is not present.

### BRIEF DESCRIPTION OF DRAWINGS

FIG. 1 is a perspective view illustrating an example of an external appearance of a sample analyzer in accordance with one or more embodiments.

FIG. 2 is a perspective view illustrating an example of a main part of a sample analyzer in accordance with one or more embodiments.

FIG. 3 is a perspective view illustrating an example of a configuration of a sample processing unit.

FIG. 4 is a perspective view illustrating an example of a configuration of a testing unit.

FIG. 5 is a perspective view illustrating an example of a main part of the testing unit.

FIG. 6 is a cross-sectional view of an irradiation part along section plane SP1 in FIG. 5.

FIG. 7 is a cross-sectional view of the irradiation part along section plane SP2 in FIG. 5.

FIG. 8 is a plan view illustrating an example of a configuration of a test piece.

FIG. 9 is a side view illustrating an example of a configuration of a test piece.

FIG. 10 is a graph illustrating a relationship between an accumulation ratio and a brightness value on a measurement surface on an indicator section.

FIG. 11 is a side view illustrating a state of a sample supplied onto an indicator section.

FIG. 12 is a graph illustrating a relationship between an accumulation ratio and a brightness value on a measurement surface on an indicator section.

FIG. 13 is a graph illustrating a relationship between an accumulation ratio and a brightness value on a measurement surface on an indicator section.

FIG. 14 is a graph illustrating a relationship between a accumulation ratio and a brightness value on a measurement surface on an indicator section.

FIG. 15 is a graph illustrating a relationship between a decrease rate and a standard deviation of a brightness distribution on a measurement surface on an indicator section.

FIG. 16 is a block diagram illustrating an example of a configuration of a control unit.

FIG. 17 is a flowchart for explaining a procedure for determining a measurement item associated with each indicator section.

### EMBODIMENTS

With reference to the drawings, an embodiment is described in detail below.

### 1. Configuration of Sample Analyzer

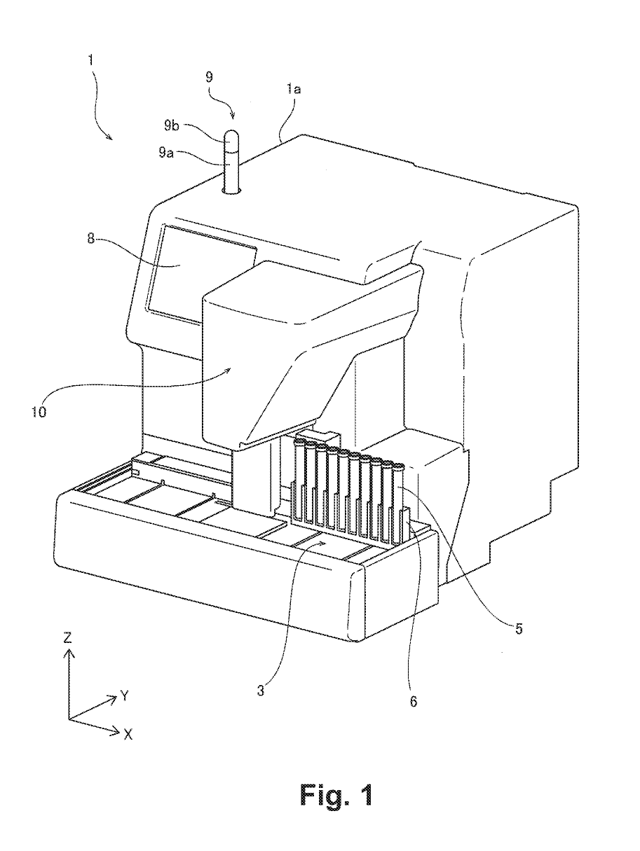

FIG. 1 is a perspective view illustrating an example of an external appearance of sample analyzer 1 in accordance with one or more embodiments. Here, sample analyzer 1 is a device for testing a liquid sample such as urine for concentrations of components (for example, glucose, protein, and the like) contained in the sample and the specific gravity of the sample. As illustrated in FIG. 1, transport unit 3 is provided in a front part of sample analyzer 1. Also, housing 1a of sample analyzer 1 is mainly provided with display unit 8 and warning light 9.

Note that an XYZ orthogonal coordinate system with a Z-axis direction being a vertical direction and an XY plane being a horizontal plane is attached, as needed, to each of FIGS. 1 to 9 and 11, in order to facilitate the understanding of the constituent components illustrated therein.

Transport unit 3 transports one or more Spitz tubes 5 from a loading position of transport stage 6 to an unloading position of transport stage 6 through a collection position below sample processing unit 10. Here, each of Spitz tubes 5 is a storage part that stores a sample. As illustrated in FIG. 1, Spitz tubes 5 are transported in a state of being propped up on transport stage 6.

Display unit 8 includes a liquid crystal display, for example, and has a function of “touch panel” that enables specification of a position on a screen by touching the screen with a finger or a dedicated pen. Therefore, a user of sample analyzer 1 (hereinafter simply referred to as the “user”) can cause sample analyzer 1 to execute predetermined processing (for example, to start analyzing the sample stored in Spitz tube 5) by giving instructions using the “touch panel” function of display unit 8 based on contents displayed on display unit 8. Thus, display unit 8 can be used as an input unit that receives an input operation from the user.

Warning light 9 is a notification unit for notifying the user of an operational status of sample analyzer 1, and is made of a transparent body such as an acrylic bar, for example. As illustrated in FIG. 1, warning light 9 includes transparent main body part 9a and lighting part 9b with a surface made opaque by sanding or the like. Thus, when light is guided to warning light 9 from an end of the main body part 9a, lighting part 9b emits light of a color depending on the guided light.

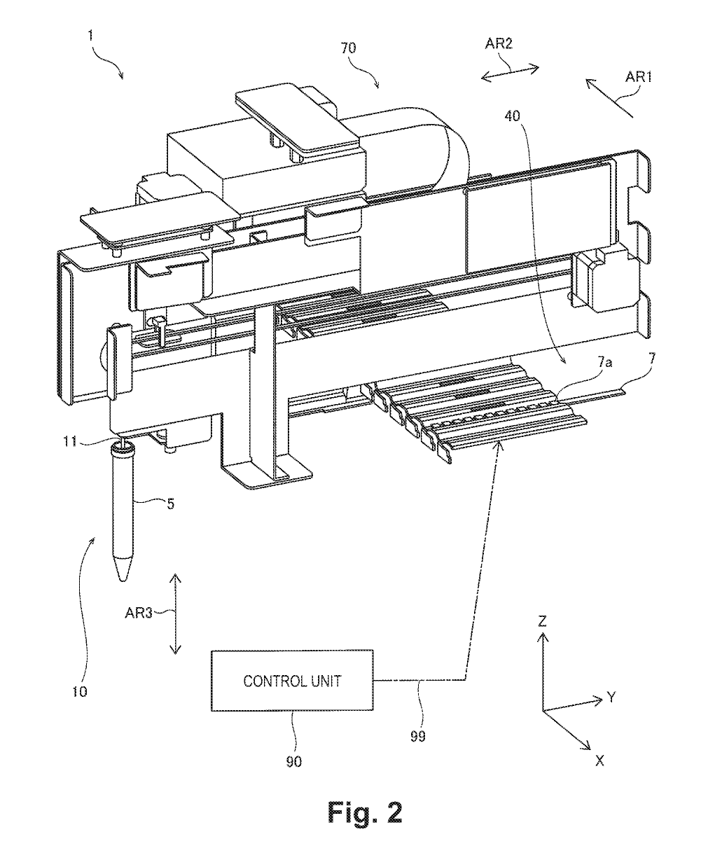

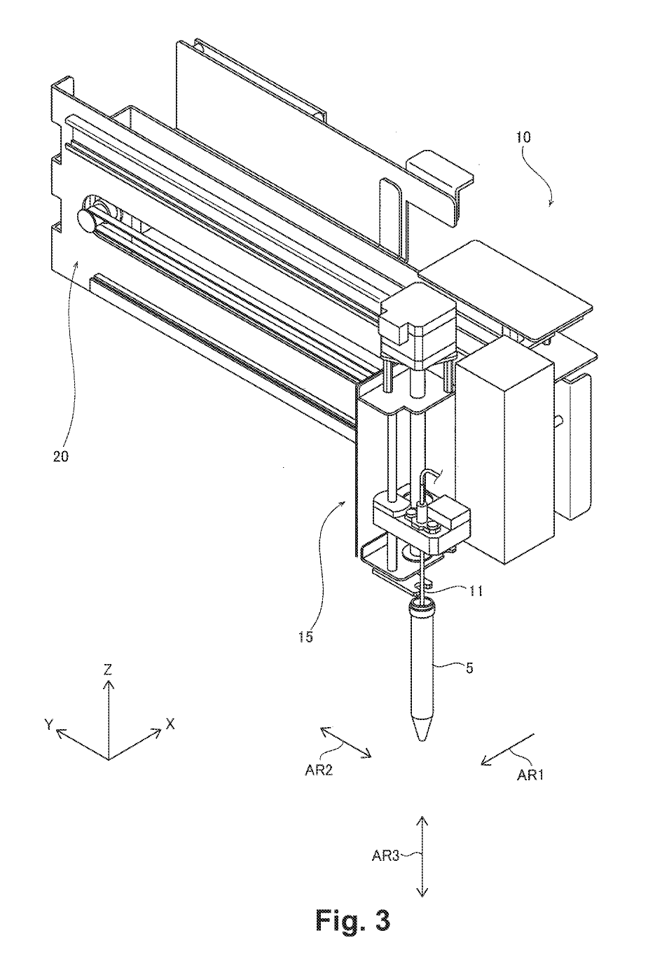

FIG. 2 is a perspective view illustrating an example of a main part of sample analyzer 1. FIG. 3 is a perspective view illustrating an example of a configuration of sample processing unit 10. As illustrated in FIG. 2, sample analyzer 1 mainly includes sample processing unit 10, transfer unit 40, testing unit 70, and control unit 90.

Sample processing unit 10 delivers the sample aspirated into nozzle 11 from Spitz tube 5 to a desired position (for example, each of indicator sections 7a of test piece 7 placed in transfer unit 40, or the like). As illustrated in FIG. 3, sample processing unit 10 mainly includes nozzle 11, elevator 15, and advance/retreat part 20.

Nozzle 11 is a cylindrical body made of a conductive material. An aspirated sample or cleaning liquid can be delivered from a tip of nozzle 11. Elevator 15 moves nozzle 11 in an up-and-down direction (arrow AR3 direction) with respect to a liquid level of the sample stored in Spitz tube 5. Advance/retreat part 20 moves elevator 15 in a back-and-forth direction (arrow AR2 direction) between a position above Spitz tube 5 and a position above transfer unit 40.

Therefore, nozzle 11 can be moved between a sample aspiration position and a delivery position where the sample is delivered onto test piece 7 by driving elevator 15 and advance/retreat part 20, as illustrated in FIG. 2.

Transfer unit 40 transfers test piece 7 with the sample supplied in indicator sections 7a from sample processing unit 10 to testing unit 70. As illustrated in FIG. 2, a transfer direction (arrow AR1 direction) of test piece 7 is approximately orthogonal to the back-and-forth direction (arrow AR2 direction) of nozzle 11.

Testing unit 70 executes determination of a measurement item associated with each indicator section 7a by taking an image of indicator section 7a of test piece 7 and performing image processing on the acquired imaging data. Note that the configuration of testing unit 70 is described in detail later.

Control unit 90 is electrically connected to sample processing unit 10, transfer unit 40, and testing unit 70 through signal line 99. Control unit 90 controls operations of sample processing unit 10, transfer unit 40, and testing unit 70. Note that the configuration of control unit 90 is described in detail later.

### 2. Configuration of Testing Unit

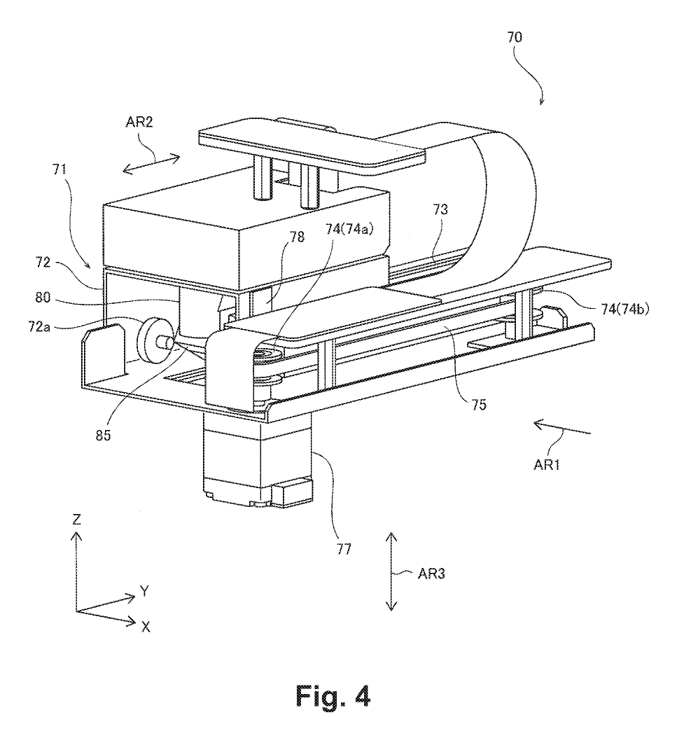

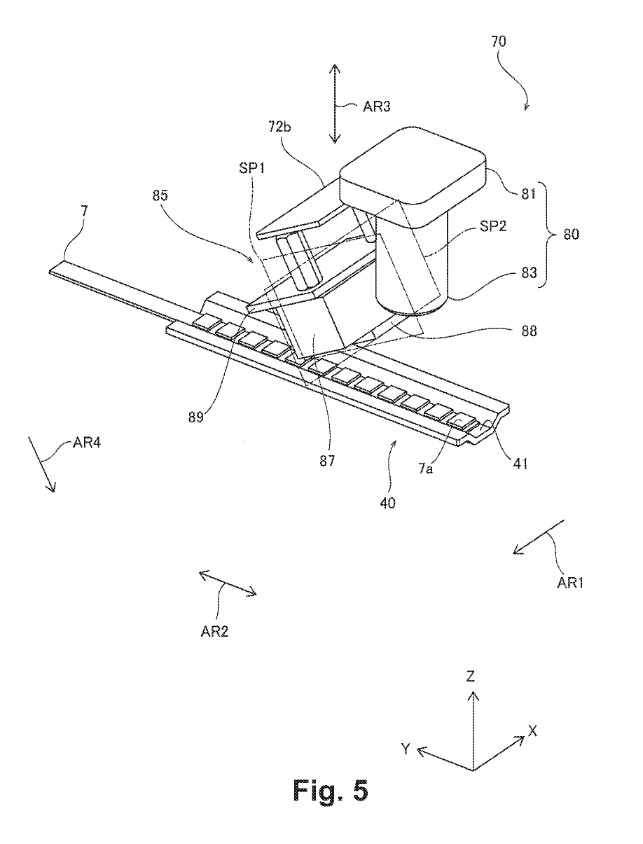

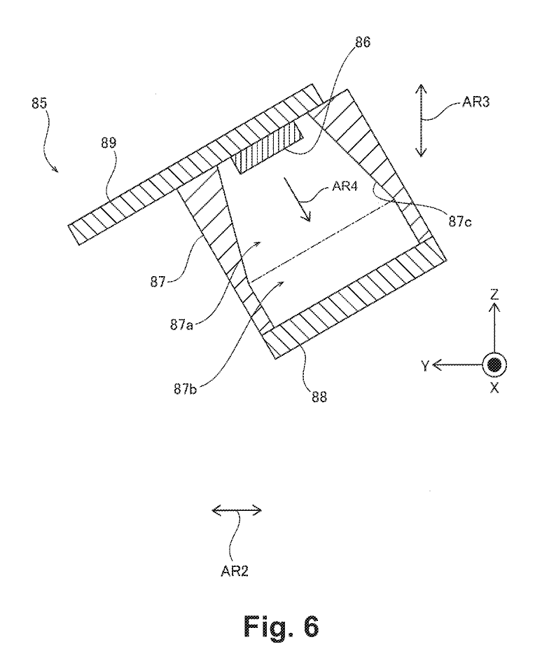

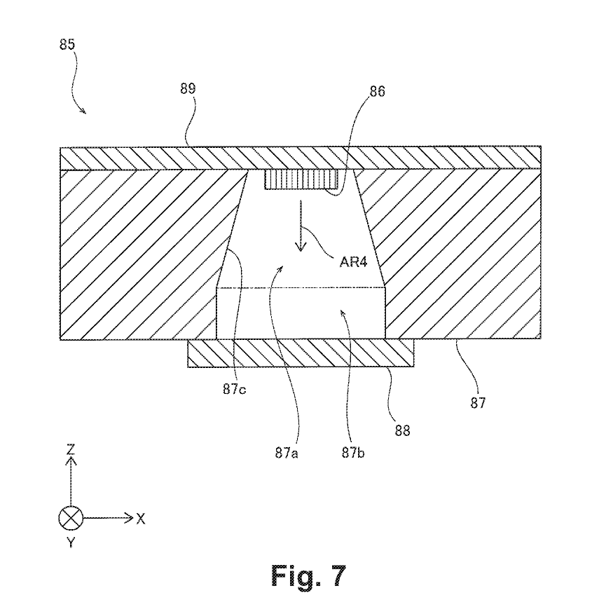

FIG. 4 is a perspective view illustrating an example of the configuration of testing unit 70. FIG. 5 is a perspective view illustrating an example of a main part of testing unit 70. FIG. 6 is a cross-sectional view of irradiation part 85 along section plane SP1 in FIG. 5. FIG. 7 is a cross-sectional view of irradiation part 85 along section plane SP2 in FIG. 5.

Testing unit 70 tests a liquid sample based on a brightness distribution in indicator section 7a provided in test piece 7. As illustrated in FIG. 4, testing unit 70 mainly includes shifting part 71, imaging part 80, and irradiation part 85.

Here, “brightness” represents the lightness of an object surface. In this embodiment, the values of pixels in an image taken by imaging part 80 serve as brightness values, and a distribution of the values of the pixels (that is, imaging data) serves as the brightness distribution. When imaging part 80 takes an RGB (Red, Green, and Blue) color image, for example, three brightness distributions can be acquired based on R pixels, G pixels, and B pixels.

Shifting part 71, or a shifting device, moves imaging part 80 and irradiation part 85 in an arrangement direction (arrow AR2 direction) of indicator sections 7a during imaging. In other words, shift part 71 is configured to move imaging part 80 and irradiation part 85 in a longitudinal direction of test piece 7. As illustrated in FIG. 4, shifting part 71 mainly includes fixing frame 72, guide 73, pulleys 74 (74a and 74b), belt 75, transfer motor 77, and fixture 78.

Fixing frame 72 is a frame body for fixing imaging part 80 and irradiation part 85. As illustrated in FIG. 4, wheel 72a is provided in a lower part of fixing frame 72. Also, fixing frame 72 is slidable on guide 73 extending in a back-and-forth direction (arrow AR2 direction).

Pulleys 74 (74a and 74b) rotates about its axial center approximately parallel to the arrow AR3 direction. Also, belt 75 is wound around the outer circumference of pulleys 74 (74a and 74b). Moreover, the axial center of pulley 74a is connected to a rotation axis of transfer motor 77. Furthermore, shifting part 71 is fixed to belt 75 with fixture 78.

Thus, shifting part 71 is moved in the back-and-forth direction (arrow AR2 direction) by rotating transfer motor 77 in a positive direction or a negative direction. Thus, imaging part 80 can be moved to immediately above indicator section 7a.

Imaging part 80, or an imaging device, takes an image of each indicator section 7a by being moved in the back-and-forth direction by shifting part 71. More specifically, imaging part 80 obtains images of reference surface 7d and measurement surface 7c on test piece 7. As illustrated in FIG. 5, imaging part 80 mainly includes: imaging element 81 or a sensor; and lens system 83 or a lens.

Lens system 83 focuses light reflected by test piece 7, for example, onto imaging element 81. As illustrated in FIG. 5, imaging part 80 is fixed to fixing frame 72 (see FIG. 4) such that an optical axis of lens system 83 is parallel to arrow AR3.

Imaging element 81 includes light receiving elements, and converts the light focused by lens system 83 into an electric signal depending on the intensity of the light. Here, a CCD (Charge Coupled Device) image sensor or a CMOS (Complementary Metal-Oxide Semiconductor) image sensor, for example, may be employed as imaging element 81. Alternatively, one having light receiving elements one-dimensionally or two-dimensionally arranged therein may be employed as imaging element 81. Still alternatively, one capable of acquiring a gray-scale image or a color image may be employed as imaging element 81.

Irradiation part 85, or an irradiation device, irradiates indicator section 7a in test piece 7 with diffused light, for example. More specifically, irradiation part 85 irradiates reference surface 7d and measurement surfaces 7c on test piece 7 with light. As illustrated in FIGS. 5 and 6, irradiation part 85 is fixed to mounting body 72b (see FIG. 5) of fixing frame 72 such that a radiation direction (arrow AR4 direction: see FIG. 6) of the light applied from irradiation part 85 is tilted with respect to the optical axis (parallel to the arrow AR3 direction) of lens system 83. As illustrated in FIG. 5, irradiation part 85 mainly includes light source 86, light guide 87, and diffuser 88. Here, the “radiation direction” means a direction parallel to the central axis of the diffused light applied by irradiation part 85.

Light source 86 is a point light source including LEDs (Light Emitting Diodes), for example. As illustrated in FIG. 6, light source 86 is fixed to plate-like cover 89 disposed on light guide 87.

Diffuser 88 transmits light emitted from light source 86 therethrough, thereby converting the emitted light into diffused light. As illustrated in FIGS. 6 and 7, diffuser 88 is provided below light guide 87 so as to close straight advance hole 87b. Here, as diffuser 88, a translucent white acrylic plate or an acrylic plate with a surface made opaque by sanding or the like may be employed, for example.

Light guide 87 guides the light emitted from light source 86 to diffuser 88. As illustrated in FIGS. 6 and 7, light guide 87 is substantially a cylindrical body having light guiding hole 87a and straight advance hole 87b formed therein along the radiation direction, which is provided between light source 86 and diffuser 88. Thus, the light emitted from light source 86 is guided to diffuser 88 through light guiding hole 87a and straight advance hole 87b.

Moreover, as illustrated in FIGS. 6 and 7, the longer the distance from light source 86, the larger the inner perimeter (a length along inner peripheral wall 87c of light guide 87) of light guiding hole 87a in a direction orthogonal to the radiation direction (arrow AR4 direction) of the diffused light. More specifically, light guiding hole 87a has its width increased toward diffuser 88 from light source 86.

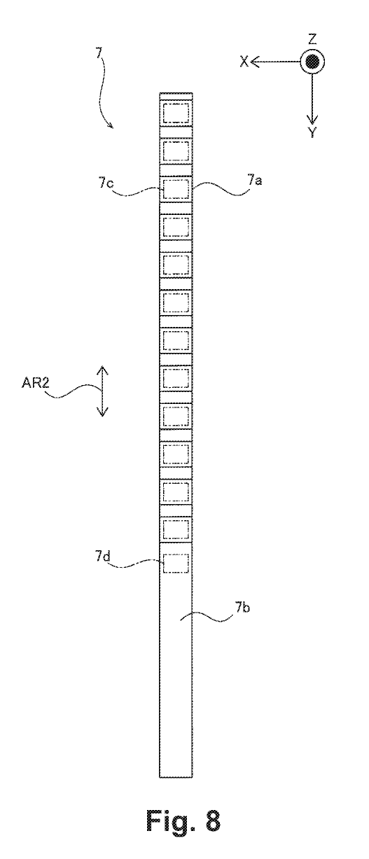

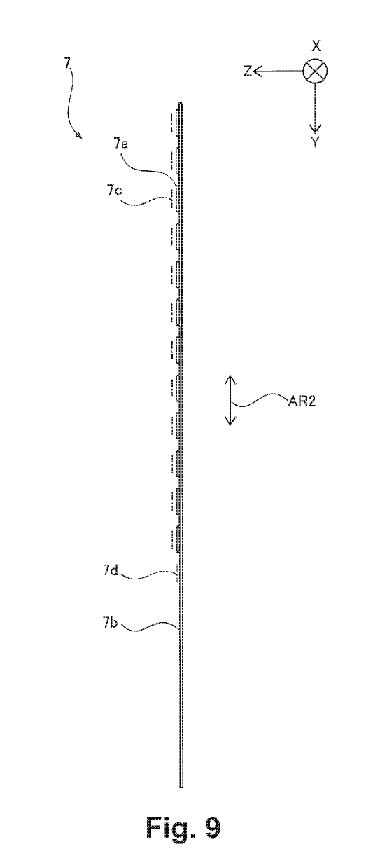

As described above, imaging part 80 and irradiation part 85 in testing unit 70 are moved relative to test piece 7 placed on installation part 41 in transfer unit 40. Therefore, imaging part 80 can acquire imaging data concerning respective measurement surfaces 7c (see FIGS. 8 and 9) on test piece 7 while being moved by shifting part 71.

FIG. 8 is a plan view and FIG. 9 is a side view illustrating an example of a configuration of test piece 7. Here, test piece 7 is a test strip for qualitatively or quantitatively measuring the concentrations of components dissolved in a liquid sample. As illustrated in FIGS. 8 and 9, test piece 7 mainly includes indicator sections 7a and base section 7b. Indicator sections 7a comprises a reagent or a substance configured to react with components in a sample. Color or brightness of indicator section 7a is changed by the reaction of the reagent or the substance with the components, and indicator section 7a can provide indication on the components in the sample. Note that indicator sections 7a may not include a substance configured to react with components in a sample. In this case, indicator section 7a may indicate color or brightness of a sample that is supplied onto indicator section 7a.

Indicator sections 7a are associated with specific measurement items (for example, specific components dissolved in the sample), respectively. As illustrated in FIGS. 8 and 9, indicator sections 7a are arranged in a longitudinal direction (arrow AR2 direction) of base section 7b.

When the liquid sample is supplied onto measurement surface 7c on each indicator section 7a, brightness on each measurement surface 7c varies depending on the concentration of the associated measurement item (component). To be more specific, when the sample is supplied onto measurement surface 7c on indicator section 7a, the brightness on the entire or a part of measurement surface 7c is decreased depending on the sample absorption condition of measurement surface 7c and the components in the sample.

Base section 7b is a support for disposing indicator sections 7a, and the color of base section 7b is set to one with a high brightness (for example, white). In determination of each measurement item, an index of brightness on reference surface 7d on base section 7b is used as a reference value of brightness.

### 3. Method of Determining Measurement Item

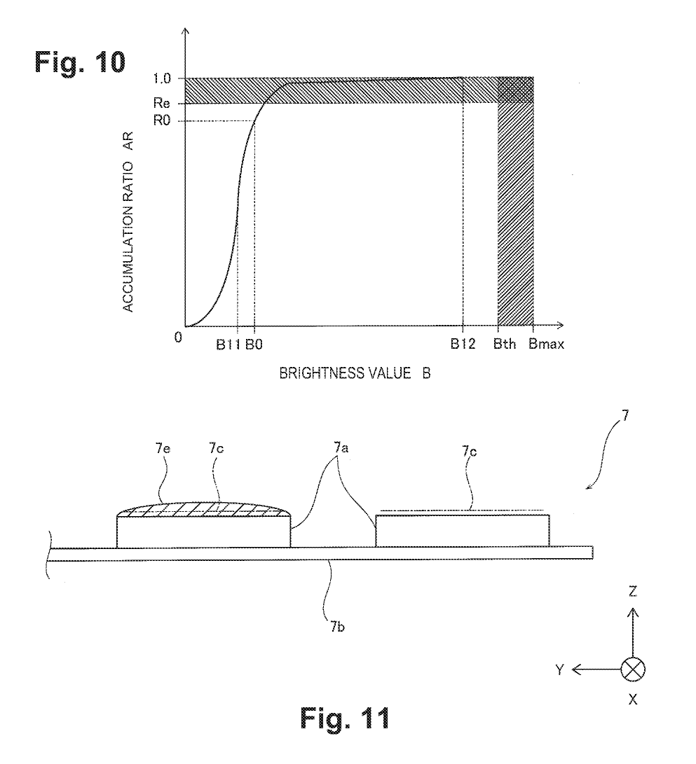

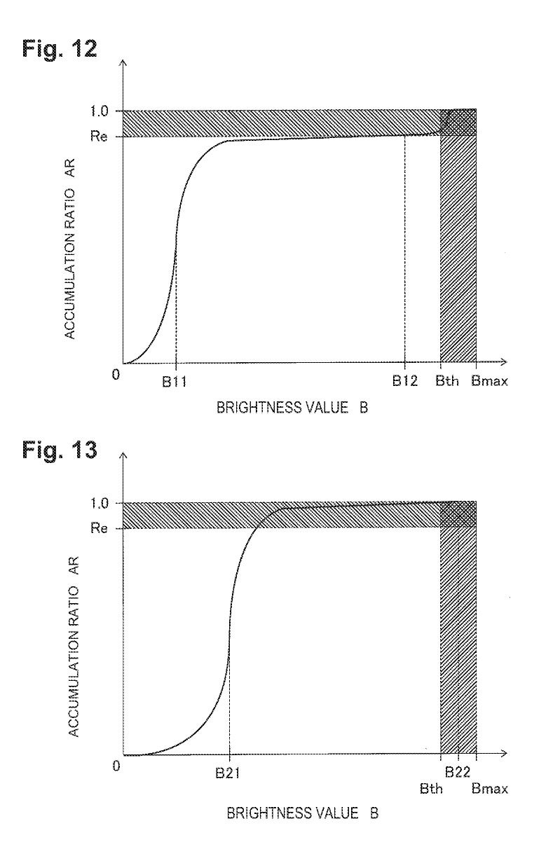

FIG. 10 is a graph illustrating a relationship between an accumulation ratio and a brightness value on measurement surface 7c on indicator section 7a. FIG. 11 is a side view illustrating a state of sample 7e supplied onto indicator section 7a. As in the case of FIG. 10, FIGS. 12 to 14 are graphs each illustrating a relationship between the accumulation ratio and the brightness value on measurement surface 7c on indicator section 7a. FIG. 15 is a graph illustrating a relationship between a decrease rate and a standard deviation of a brightness distribution on measurement surface 7c on indicator section 7a. Hereinafter, a method of determining a measurement item associated with indicator section 7a is described after description of the brightness distribution on measurement surface 7c.

Here, in FIGS. 10 and 12 to 14, the vertical axis represents the accumulation ratio and the horizontal axis represents the brightness value. When it is defined that the total number of pixels within measurement surface 7c is Ca and the number of pixels having the brightness value “B”=“0” to “BO” within the measurement surface 7c is C0, the accumulation ratio “AR”=“R0” at the brightness value “B”=“BO” is calculated by Equation (1).

R0=C0/Ca (1)

Moreover, decrease rate D or a change rate used for the method of determining a measurement item is obtained as follows. Specifically, when it is defined that an average value of a brightness distribution on reference surface 7d of base section 7b is BSave and an average value of a brightness distribution on measurement surface 7c is Bave, decrease rate D is calculated by Equation (2).

D=Bave/BSave (2)

Note that, when one capable of acquiring an RGB color image is employed as imaging element 81 in imaging part 80, the brightness distributions used in Equation (2) may be based on any of R pixels, G pixels, and B pixels or may be based on a gray scale image obtained by converting the RGB image.

Furthermore, in the following description, the average value of the brightness distribution on reference surface 7d is referred to as “reference brightness” and the average value of the brightness distribution on measurement surface 7c is referred to as “first sample brightness”.

First, description is given of a brightness distribution on normal measurement surface 7c. FIG. 10 is a graph illustrating a relationship between an accumulation ratio and a brightness value when the sample supplied onto measurement surface 7c is all absorbed by indicator section 7a (illustrated as indicator section 7a on the right side of FIG. 11).

FIG. 10 shows the following. Specifically, the average value of the brightness distribution of the pixels having the brightness decreased by the sample within measurement surface 7c is around “B11”, and the brightness values “B” of the pixels without any decrease in brightness within measurement surface 7c are around “B12”.

Next, description is given of a brightness distribution when imaging data on measurement surface 7c includes a specular reflection region. FIG. 12 is a graph illustrating a relationship between an accumulation ratio and a brightness value when a part of the sample supplied onto measurement surface 7c remains on indicator section 7a (illustrated as indicator section 7a on the left side of FIG. 11).

FIG. 12 shows the following. Specifically, light is regularly reflected on sample 7e present as a droplet on indicator section 7a, and brightness values “B” of pixels corresponding to a region where the light is regularly reflected (hereinafter simply referred to as the “specular reflection region”) are within a range of “Bth” to “Bmax”. Moreover, as in the case of FIG. 10, the average value of the brightness distribution of the pixels having the brightness decreased by the sample within measurement surface 7c is around “B11”, and the brightness values “B” of the pixels without any decrease in brightness within measurement surface 7c are around “B12”.

As described above, imaging data concerning the specular reflection region is obtained by taking an image of the light regularly reflected on the droplet. In this imaging data, an image of measurement surface 7c that should normally be taken is not properly recorded. Therefore, in order to properly perform determination of a measurement item associated with each indicator section 7a, the specular reflection region needs to be removed from the imaging data.

Here, examples of a method of removing pixels corresponding to the specular reflection region include

(1) a method of removing all pixels with the accumulation ratio “AR”≧“Re” from the imaging data and

(2) a method of removing all pixels with the brightness value “B”≧“Bth” from the imaging data.

However, the following problem arises when the method (1) is employed. Specifically, when there is no specular reflection region as in the case of FIG. 10, a region with the accumulation ratio “AR”≧“Re” should normally be used for calculation of decrease rate D by Equation (2). As a result, removing the region with the accumulation ratio “AR”≧“Re” causes a problem that decrease rate D cannot be properly calculated and determination of the associated measurement item cannot be properly performed.

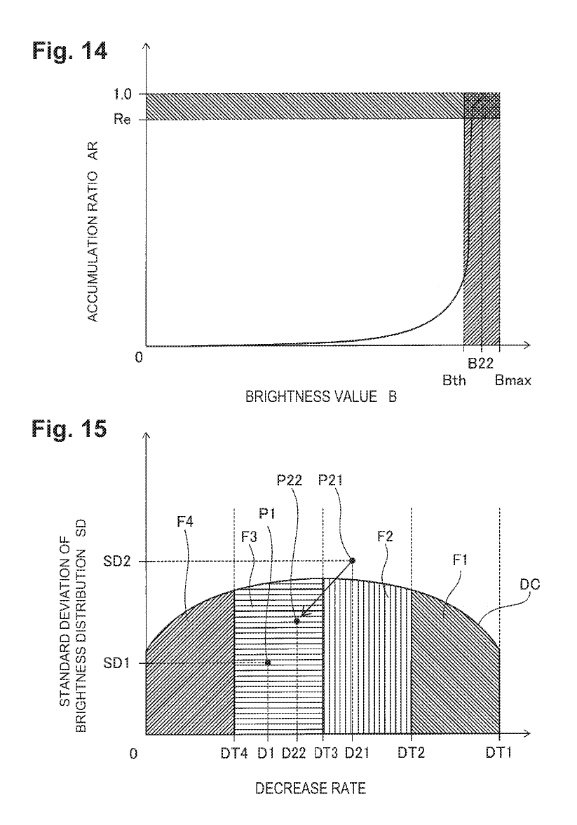

On the other hand, the method (2) is effective when the brightness values “B” of the pixels without any decrease in brightness within measurement surface 7c are smaller than “Bth” as in the case of FIGS. 10 and 12. However, the following problem arises in the case of brightness distributions illustrated in FIGS. 13 and 14.

Here, FIG. 13 is the same as FIG. 10 in terms of illustrating a relationship between an accumulation ratio and a brightness value when the sample supplied onto measurement surface 7c is all absorbed by indicator section 7a (illustrated as indicator section 7a on the right side of FIG. 11). On the other hand, FIG. 13 is different from FIG. 10 in that the brightness value “B”=“B22” of the pixel without any decrease in brightness within measurement surface 7c is included in the range of “Bth” to “Bmax”.

Therefore, when the method (2) is employed, the region that should normally be used for calculation of decrease rate D by Equation (2) is removed if the brightness on measurement surface 7c is not decreased even when the sample is supplied onto indicator section 7a, for example (see FIG. 14). As a result, there arises a problem that decrease rate D cannot be properly calculated and determination of the associated measurement item cannot be properly performed, as in the case of the method (1).

Therefore, in this embodiment, focusing attention on a relationship between a standard deviation of a brightness distribution on measurement surface 7c and a decrease rate, a determination method that solves the problems of the methods (1) and (2) is employed. Specifically, a standard deviation of a brightness distribution when there is a specular reflection region (case of FIG. 12) is larger than that when there is no specular reflection region (case of FIG. 10).

Determining the magnitude of the standard deviation of the brightness distribution as described above makes it possible to determine whether or not the imaging data includes a specular reflection region. In order to enable this determination, determination curves DC illustrated in FIG. 15 are employed for the respective components contained in the sample in this embodiment.

For example, as illustrated in FIG. 15, when a plotted point of the calculated standard deviation and decrease rate (first decrease rate or first change rate) is below determination curve DC (hatched region) such as point P1 (D1, SD1), it is determined that the imaging data includes no specular reflection region. Then, determination of a measurement item (concentration of the associated component) is executed based on decrease rate (D1) (first decrease rate) at point P1.

Here,

(A) as illustrated in FIG. 15, ranges of decrease rates “DT2”<“D”≦“DT1”, “DT3”<“D”≦“DT2”, “DT4”<“D”≦“DT3”, and “0”≦“D”≦“DT4” are defined as regions F1 to F4, respectively, and

(B) regions F1 to F4 are previously empirically defined as corresponding to component concentration ranges A1 to A4, respectively. In this case, the decrease rate “D”=“D1” is included in region F3 as illustrated in FIG. 15. Therefore, the concentration of the measurement item (component) associated with indicator section 7a is determined to be included in concentration range A3.

On the other hand, when a plotted point of the standard deviation and decrease rate calculated from the imaging data concerning measurement surface 7c is on or above determination curve DC such as point P21 (D21, SD2), it is determined that the imaging data includes a specular reflection region. Then, determination of a measurement item is executed based on a second sample brightness in a remnant region of the imaging data concerning measurement surface 7c from which the specular reflection region is excluded.

To be more specific, when it is determined that the imaging data includes a specular reflection region, a remnant region is set by excluding pixels with the brightness value “B” within the range of “Bth” to “Bmax” from the imaging data concerning measurement surface 7c. Next, an average value of brightness in the remnant region is calculated as the second sample brightness, and the decrease rate “D”=“D22” is calculated, using Equation (2), from the second sample brightness and the reference brightness.

The determination of the measurement item is executed based on the decrease rate “D22” (second decrease rate or second change rate) at point P22 rather than point P21. Specifically, the decrease rate “D”=“D22” is included in region F3 rather than region F2 as illustrated in FIG. 15. Therefore, the concentration of the measurement item (component) associated with indicator section 7a is determined to be included in concentration range A3 rather than concentration range A2.

Note that determination curve DC may be previously obtained by experiment or the like for each of the components contained in the sample or may be obtained using a predetermined calculation formula.

### 4. Procedure for Determining Measurement Item by Testing Unit

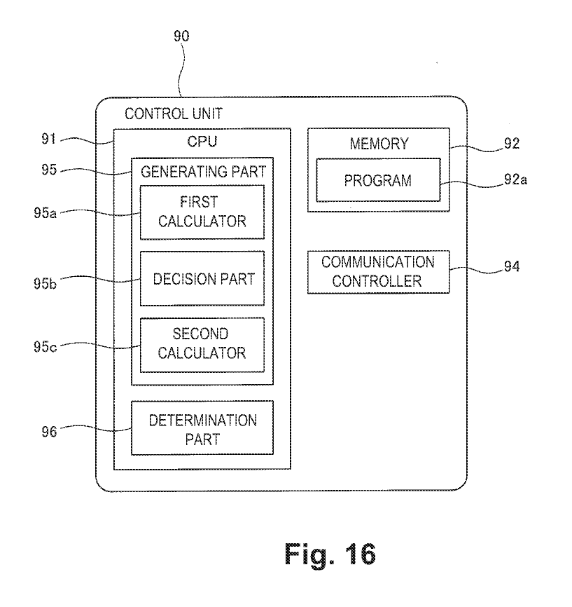

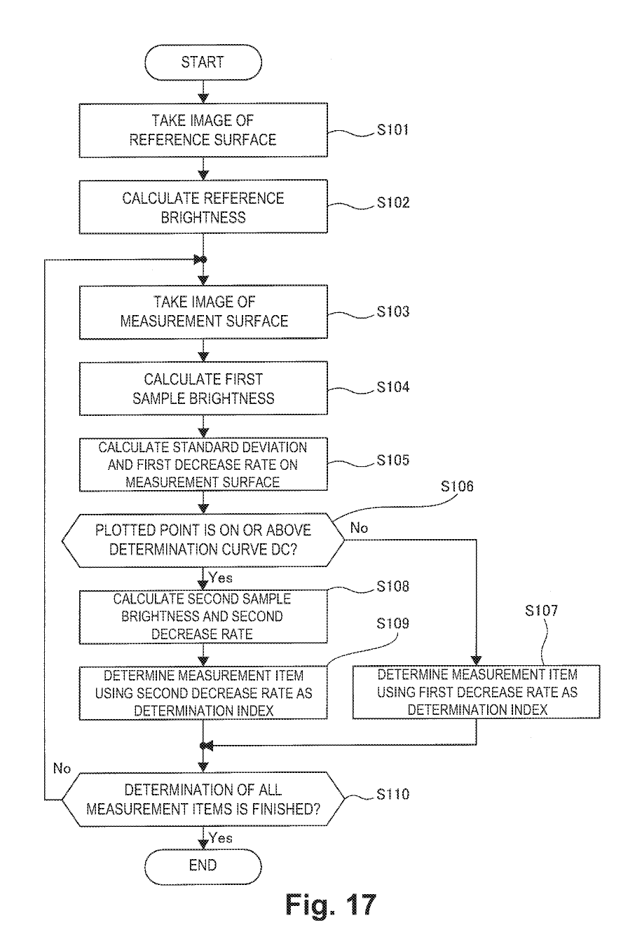

FIG. 16 is a block diagram illustrating an example of a configuration of control unit 90. FIG. 17 is a flowchart for explaining a procedure for determining a measurement item associated with each indicator section 7a. As illustrated in FIG. 16, control unit 90 mainly includes CPU 91, memory 92, and communication controller 94.

CPU (Central Processing Unit) 91 executes operation control and data processing in accordance with program 92a in memory 92. Also, calculation functions executed by blocks (denoted by reference numerals 95 (95a, 95b, and 95c) and 96) in CPU 91 in FIG. 16 are implemented by CPU 91.

Generating part 95 generates a determination index to be used by determination part 96, based on imaging data acquired by imaging part 80. As illustrated in FIG. 16, generating part 95 mainly includes first calculator 95a, decision part 95b, and second calculator 95c.

First calculator 95a calculates a reference brightness as an index of brightness on reference surface 7d (see FIGS. 8 and 9) set on test piece 7, and also calculates a first sample brightness as an index of brightness on measurement surface 7c.

Decision part 95b uses Equation (2) to calculate a first decrease rate that is a ratio of the first sample brightness to the reference brightness. Also, decision part 95b determines whether or not imaging data concerning measurement surface 7c includes a specular reflection region, based on a relationship (see FIG. 15) between a standard deviation of a brightness distribution on measurement surface 7c and the first decrease rate.

When it is determined by decision part 95b that the specular reflection region is present, second calculator 95c calculates a second sample brightness as an index of brightness in a remnant region of measurement surface 7c from which the specular reflection region is excluded. Furthermore, second calculator 95c calculates a second decrease rate that is a ratio of the second sample brightness to the reference brightness. Here, as the remnant region, part of the imaging data from which data with the brightness value “B” not less than a threshold “Bth” is excluded may be employed, as illustrated in FIG. 12.

Determination part 96 execute determination on the measurement item associated with measurement surface 7c using the determination index generated by generating part 95. More specifically, determination part 96 determines quality or quantity in terms of the measurement item corresponding to measurement surface 7c. For example, when it is determined by decision part 95b that there is a specular reflection region on measurement surface 7c, determination part 96 uses the determination index (for example, the second decrease rate) based on the second sample brightness to determine the measurement item associated with measurement surface 7c.

On the other hand, when it is determined by decision part 95b that there is no specular reflection region, determination part 96 uses the determination index (for example, the first decrease rate) based on the first sample brightness to determine the measurement item associated with measurement surface 7c.

Communication controller 94 can transmit control signals to transfer motor 77, imaging element 81, light source 86, and the like connected through signal line 99 (see FIG. 2). Thus, communication controller 94 can operate transfer motor 77, imaging element 81, light source 86, and the like at predetermined timing.

Next, with reference to FIG. 17, description is given of a procedure for determining a measurement item associated with each indicator section 7a. Note that, prior to Step S101, test piece 7 to be tested is moved to immediately below imaging part 80 and irradiation part 85 by transfer unit 40. Also, imaging part 80 and irradiation part 85 are moved to immediately above reference surface 7d by shifting part 71.

In this determination procedure, first, imaging part 80 takes an image of reference surface 7d of test piece 7 (S101) and a reference brightness is calculated using imaging data thus obtained (S102).

Subsequently, imaging part 80 and irradiation part 85 are moved to immediately above measurement surface 7c adjacent to reference surface 7d, and an image of measurement surface 7c is taken (S103). Then, a first sample brightness is calculated based on imaging data thus obtained (S104). Thereafter, a standard deviation of a brightness distribution on measurement surface 7c and a first decrease rate are calculated (S105).

Here, when it is determined that a point (D, SD) plotted on the graph of FIG. 15 is on or above determination curve DC (S106), decision part 95b determines that the imaging data on measurement surface 7c includes a specular reflection region. Then, after a second sample brightness and a second decrease rate are calculated (S108), the second decrease rate is used as a determination index to determine the measurement item (S109).

On the other hand, when it is determined that a point (D, SD) plotted on the graph of FIG. 15 is below determination curve DC (S106), decision part 95b determines that the imaging data on measurement surface 7c includes no specular reflection region. Then, the first decrease rate is used as a determination index to determine the measurement item (S107).

In this determination procedure, Steps S103 to S109 are executed until determination of all the measurement items to be measured is finished (S110).

### 5. Advantages of Testing Unit According to Embodiment

As described above, testing unit 70 according to this embodiment determines the measurement item associated with indicator section 7a by

(1) using the determination index (that is, the second decrease rate) based on the second sample brightness, when it is determined that the imaging data includes a specular reflection region, and

(2) using the determination index (that is, the first decrease rate) based on the first sample brightness, when it is determined that the imaging data includes no specular reflection region.

Therefore, even when the imaging data includes a specular reflection region, the specular reflection region can be successfully removed while leaving a region to be used for calculation of the determination index. Thus, erroneous determination of the measurement item attributable to the specular reflection region can be prevented.

Moreover, the light emitted from light source 86 reaches diffuser 88 while being reflected on inner peripheral wall 87c of straight advance hole 87b and light guiding hole 87a having its width increased toward diffuser 88 from light source 86. This makes it possible to successfully irradiate indicator section 7a of test piece 7 with the diffused light from irradiation part 85, and to inhibit a phenomenon in which light specularly reflected on a droplet on indicator section 7a enters imaging part 80. Therefore, the number of determinations based on the second sample brightness can be reduced. Also, calculation cost required for the determination of the measurement items, that is, processing time and calculation hardware cost can be reduced.

### 6. Modified Example

While an embodiment is described above, the invention is not limited to the embodiment described above, and various modifications can be made.

(1) In the embodiment described above, the description is given assuming that shifting part 71 moves imaging part 80 and irradiation part 85 relative to test piece 7. In one or more embodiments, for example, test piece 7 may be moved relative to imaging part 80 and irradiation part 85, or test piece 7, imaging part 80, and irradiation part 85 may be moved, respectively. Specifically, shifting part 71 moves imaging part 80 and irradiation part 85 relative to test piece 7 in the arrangement direction of indicator sections 7a during imaging.

(2) Moreover, in the embodiment described above, the description is given assuming that first calculator 95a calculates the average value of the brightness distribution on reference surface 7d as the reference brightness and calculates the average value of the brightness distribution on measurement surface 7c as the first sample brightness. In one or more embodiments, for example, the reference brightness may be a median of the brightness distribution on reference surface 7d. Also, the first sample brightness may be a median of the brightness distribution on measurement surface 7c.

Likewise, the description is given assuming that second calculator 95c calculates the average value of the brightness distribution in the remnant region of measurement surface 7c from which the specular reflection region is excluded as the second sample brightness. In one or more embodiments, for example, the second sample brightness may be a median of the brightness distribution in the remnant region of measurement surface 7c from which the specular reflection region is excluded.

(3) Moreover, in the embodiment described above, the description is given assuming that the determination of whether or not the imaging data includes the specular reflection region is executed based on the relationship (see FIG. 15) between the standard deviation of the brightness distribution on measurement surface 7c and the first decrease rate. In one or more embodiments, for example, the determination of whether or not there is a specular reflection region may be executed based on a relationship between a dispersion of the brightness distribution on measurement surface 7c and the first decrease rate.

(4) Furthermore, in the embodiment described above, the description is given assuming that generating part 95 and determination part 96 are realized in a software manner by CPU 91 based on the program stored in memory 92. In one or more embodiments, for example, generating part 95 and determination part 96 may be realized in a hardware manner using an electronic circuit.

In the case of taking an image of an indicator section where a liquid sample is supplied and testing the sample using imaging data thus obtained, the following problem arises depending on a sample absorption condition on the indicator section.

Specifically, a part of the liquid sample is not absorbed and left on the indicator section in some cases, and light emitted from a lighting unit is specularly reflected on the sample on the indicator section, and reaches an imaging unit. In this case, imaging data of a region where the light is specularly reflected is of an image where the light regularly reflected on a droplet is taken. This imaging data does not record the proper image of the measurement surface that should be taken essentially. This results in the problem that the imaging data of the specular reflection region adversely affects testing of the sample.

According to the embodiments described above, it can provide a testing unit capable of successfully testing a sample even when imaging data includes a specular reflection region, and a sample analyzer including the testing unit.

According to the embodiments described above, the measurement item associated with the indicator section is determined by

(1) using the determination index based on the second sample brightness, when it is determined that the imaging data includes a specular reflection region, and

(2) using the determination index based on the first sample brightness, when it is determined that the imaging data includes no specular reflection region.

Therefore, even when the imaging data includes a specular reflection region, the specular reflection region can be successfully removed while leaving a region to be used for calculation of the determination index. Thus, erroneous determination of the measurement item attributable to the specular reflection region can be prevented.

Particularly, in the configuration including the light source and the configuration in which the imaging part and the irradiation part are moved, light emitted from the light source reaches the diffuser while being reflected on an inner wall of a light guiding hole having its width increased toward the diffuser from the light source. This enables the indicator section to be successfully irradiated with the diffused light from the irradiation part, and inhibits a phenomenon in which light specularly reflected on a droplet on the indicator section enters the imaging part. Therefore, the number of determinations based on the second sample brightness can be reduced. Also, calculation cost required for the determination of the measurement items, that is, processing time and calculation hardware cost can be reduced.

The invention includes other embodiments in addition to the above-described embodiments without departing from the spirit of the invention. The embodiments are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

What is claimed is:

1. A testing unit to test a liquid sample applied on a measurement surface in an indicator section on a test piece by using a brightness distribution, comprising:
(a) an irradiation part that irradiates a reference surface and the measurement surface on the test piece with light;
(b) an imaging part that obtains images of the reference surface and the measurement surface on the test piece;
(c) a generating part that generates a determination index based on the imaging data obtained by the imaging part; and
(d) a determination part that executes determination, using the determination index, on a measurement item associated with the indicator section, wherein
the generating part includes:
(c-1) a first calculator that calculates a brightness, as a reference brightness, of the reference surface on the test piece, and calculates a brightness, as a first sample brightness, of the measurement surface,
(c-2) a decision part that determines whether or not the imaging data concerning the measurement surface includes a specular reflection region on the basis of a relationship between a first rate calculated as a ratio of the first sample brightness to the reference brightness and a standard deviation or dispersion of a brightness distribution on the measurement surface, and
(c-3) a second calculator that calculates a brightness, as a second sample brightness, of a remnant region of the measurement surface excluding the specular reflection region, when the decision part determines that the specular reflection region is present, and
the determination part executes the determination on the measurement item associated with the indicator section,
(i) using the determination index based on the second sample brightness, when it is determined that the specular reflection region is present, and
(ii) using the determination index based on the first sample brightness, when it is determined that the specular reflection region is not present.

2. The testing unit according to claim 1, wherein
the determination part executes the determination on the measurement item associated with the indicator section by using, as the determination index,
(i) a second rate calculated as a ratio of the second sample brightness to the reference brightness, when it is determined that the specular reflection region is present, and
(ii) the first rate when it is determined that the specular reflection region is not present.

3. The testing unit according to claim 1, wherein
when the decision part determines that the specular reflection region is present, the second calculator calculates the second sample brightness by using, as the remnant region, part of the imaging data from which data with a brightness value not less than a threshold is excluded.

4. The testing unit according to claim 1, wherein
the first calculator calculates:
an average value of a brightness distribution on the reference surface as the reference brightness and
an average value of the brightness distribution on the measurement surface as the first sample brightness, and the second calculator calculates:
an average value of a brightness distribution in the remnant region as the second sample brightness.

5. The testing unit according to claim 1, wherein
the irradiation part includes:
(a-1) a light source,
(a-2) a diffuser that transmits light emitted from the light source therethrough, thereby converting the emitted light into diffused light, and
(a-3) alight guide that is provided between the light source and the diffuser, and configured to guide the emitted light to the diffuser,
the emitted light is guided to the diffuser through a light guiding hole formed in the light guide, and
the longer the distance from the light source, the larger the inner perimeter of the light guiding hole in a direction orthogonal to a radiation direction of the diffused light.

6. The testing unit according to claim 5, wherein
the light source comprises a point light source.

7. The testing unit according to claim 1, wherein
the imaging part and the irradiation part are movable by a shifting part in a longitudinal direction of the test piece, and
the imaging part obtains the imaging data while being moved by the shifting part.

8. The testing unit according to claim 1, wherein
the sample comprises urine, and
the testing unit is configured to test a concentration of a urine component.

9. A sample analyzer comprising:
the testing unit according to claim 1;
a sample processing unit that delivers the sample onto the indicator section on the test piece; and
a transfer unit that transfers the test piece with the sample supplied onto the indicator section from the sample processing unit to the testing unit.

10. A testing method for testing a liquid sample applied on a measurement surface in an indicator section on a test piece by using a brightness distribution, comprising:
irradiating a reference surface and the measurement surface on the test piece with light;
obtaining images of the reference surface and the measurement surface on the test piece;
generating a determination index based on the imaging data obtained; and
executing determination on a measurement item associated with the indicator section by using the determination index, wherein
the generating the determination index includes:
(a) calculating a brightness, as a reference brightness, of the reference surface on the test piece, and calculating a brightness, as a first sample brightness, of the measurement surface,
(b) determining whether or not the imaging data concerning the measurement surface includes a specular reflection region on the basis of a relationship between a first rate calculated as a ratio of the first sample brightness to the reference brightness and a standard deviation or dispersion of a brightness distribution on the measurement surface, and
(c) calculating a brightness, as a second sample brightness, of a remnant region of the measurement surface excluding the specular reflection region, when it is determined that the specular reflection region is present, and
the executing the determination on the measurement item includes:
(i) executing the determination on the measurement item by using the determination index based on the second sample brightness, when it is determined that the specular reflection region is present, and
(ii) executing the determination on the measurement item by using the determination index based on the first sample brightness, when it is determined that the specular reflection region is not present.

11. The testing method according to claim 10, wherein
the executing the determination on the measurement item associated with the indicator section includes:
executing the determination on the measurement item associated with the indicator section by using, as the determination index:
a second rate calculated as a ratio of the second sample brightness to the reference brightness in the case that it is determined that the specular reflection region is present, and
the first rate in the case that it is determined that the specular reflection region is not present.

12. The testing method according to claim 10, wherein
the generating the determination index includes:
when it is determined that the specular reflection region is present, calculating the second sample brightness by using, as the remnant region, part of the imaging data from which data with a brightness value not less than a threshold is excluded.

13. The testing method according to claim 10, wherein
the generating the determination index includes:
calculating an average value of a brightness distribution on the reference surface as the reference brightness,
calculating an average value of the brightness distribution on the measurement surface as the first sample brightness, and
calculating an average value of a brightness distribution in the remnant region as the second sample brightness.

14. The testing method according to claim 10, wherein
the irradiating the reference surface and the measurement surface on the test piece with the light includes:
converting emitted light from a light source into diffused light, and irradiating the reference surface and the measurement surface on the test piece with the diffused light.

15. The testing unit according to claim 1, wherein
when the sample is supplied onto the measurement surface on the indicator section, a brightness value on the entire or a part of the measurement surface is decreased depending on a sample absorption condition of the measurement surface and components in the sample.
